(19) United States
(12) Patent Application Publication
(10) Pub. No.: US 2023/0149251 A1
(43) Pub. Date: May 18, 2023

(54) MASSAGER HAVING PERINEAL REGION MASSAGE FUNCTION, AND CONTROL METHOD THEREFOR

(71) Applicant: BODYFRIEND CO., LTD., Seoul (KR)

(72) Inventors: Woong Chul KANG, Seoul (KR); Deok Hyun KONG, Seoul (KR); Do Hyun KIM, Seoul (KR); Chul Jin JEON, Seoul (KR); Soo Hyun CHO, Seoul (KR); Ho Joon JEON, Seoul (KR)

(21) Appl. No.: 18/009,930

(22) PCT Filed: Dec. 14, 2020
(86) PCT No.: PCT/KR2020/018218

§ 371 Date: Dec. 12, 2022

(30) Foreign Application Priority Data
Jun. 12, 2020 (KR) 10-2020-0071344
Jul. 3, 2020 (KR) 10-2020-0082129

(51) Int. Cl. A61H 9/00; A61H 39/04; A61H 23/02

(52) U.S. Cl. CPC A61H 9/0078; A61H 39/04; A61H 23/0254; A61H 2201/1623; A61H 2201/0149; A61H 2205/086; A61H 2205/108; A61H 2201/0207; A61H 2201/1654; A61H 2201/1664; A61H 2203/0431; A61H 2201/1238

(57) ABSTRACT

A massager having a perineal region massage function and capable of simultaneously applying upward pressure and heat to the perineal region of a user includes: a backrest frame having an opening; a seat frame having an opening that communicates with the opening of the backrest frame; massage units for massaging the body regions of a user while moving along both sides of the backrest frame and the seat frame openings; an elastic band made from an elastic material, traversing the opening of the seat frame, and transmitting to the buttocks or thighs of the user, pressure applied from the massage units through the opening of the seat frame; a perineal region airbag provided on the elastic band and upwardly pressing the perineal region of the user while inflated by means of air supplied from the outside; and an air supply part for injecting/discharging the air into/from the perineal region airbag.

### TECHNICAL FIELD

The present invention relates to a massager, and more particularly, to a massager having a perineal region massage function capable of applying pressure and heat upward to a perineal region of a user, who is sitting on the massager, and a control method therefor.

### BACKGROUND ART

With the advancement of industry, as time of sitting on the chair rather than time of performing physical labor is gradually increased, the recognition of the significance of the chair is spread. Accordingly, various kinds of comfortable chairs based on human science have recently been developed and have been commercialized.

However, even if the chair is comfortable, in a case in which a user is sitting on the chair for a long time, since a seat portion of the chair may cause poor blood circulation by pressing the user's perineal region and prostate by a cushiony function, and thus causes various diseases, such as prostate inflammation disease, hemorrhoids, urinary incontinence, erectile dysfunction and the like. Moreover, the seat portion of the chair has been pointed out as a cause of outbreak of diseases, such as cystic diseases, vaginopathy, and the like that the groin gets sweaty in the summer season since the user's buttocks get in close contact with the seat portion of the chair to have bad ventilation.

Meanwhile, the muscles of the human body are developed while repeating contraction and relaxation, but the muscles that do not perform the contraction cannot be developed due to drooping since being left in a relaxed state. Similarly, when the perineal muscles are contracted, the related organs are developed. However, if the perineal muscles do not frequently do contraction, the perineal muscles droop and cannot be developed.

According to the reports on contraction of the perineal muscles by Dr. Kegel in U.S. and Prof. Chae Il-Bin in China, it is known that when the muscle around the perineal region is repeatedly contracted to be trained, menopausal symptoms, chronic constipation, and the like, as well as urinary incontinence, erectile dysfunction and the like, can be solved. Recently, even non-professional people widely recognize the effect of this perineal muscle contraction exercise.

According to the oriental medicine, there is an energy circulation system called a meridian in the body. If there is an abnormality in the body, the flow of the meridian stagnates somewhere, and as such, the place where the flow of the meridian is likely to stagnate is referred to as an acupuncture point. Therefore, in a case in which there is something wrong in the body or in the case in which a person wants to improve the body function, acupressure therapy has been widely used as a means for stimulating the corresponding acupoint to restore the circulation function to the normal state or to improve the function.

The acupuncture point around the perineal region among the acupuncture points is widely known as a good method to improve sexual function and to solve menopausal symptoms, chronic constipation, and the like. Additionally, recently, several devices for acupressure around such a perineal region have been developed and used. As a representative example, Korean Patent No. 10-2021729 discloses a local massage device capable of massaging the perineal region and the anus region with vibration at the same time.

A conventional massage device capable of massaging the perineal region like the registered patent includes a support member mounted at approximately central portion of a seat portion supporting a user's buttocks, and a vibrator and an acupressure member disposed on the support member so as to apply pressure to the user's perineal region by periodic vibration and acupuncture of the acupressure member by the vibrator, thereby stimulating the user's perineal region.

However, the conventional local massage device has a disadvantage in that it is impossible that a massage unit is installed in a massager designed to massage the user's back, buttocks and thighs while moving from a back portion to a thigh portion of the massager. That is, in a case in which the conventional local massage device is installed in the seat portion of the massager, the massage unit cannot move to the buttock and thigh portions or cannot massage the user's thighs and buttocks due to interference between the local massage device and the massage unit.

In addition, the conventional local massage device has several disadvantages in that it may cause inconvenience to a user since it is difficult to control pressure to the perineal region, and that it is difficult to provide pressure and heat to the perineal region.

### DISCLOSURE

### Technical Problem

Accordingly, the present invention has been made in view of the above-mentioned problems occurring in the related art, and it is an object of the present invention to provide a massager having a perineal region massage function capable of effectively applying pressure upward to a perineal region of a user and providing heat together with pressure to the perineal region, thereby maximizing perineal region massage effect, and a control method therefor.

It is another object of the present invention to provide a massager having a perineal region massage function capable of minimizing interference between a perineal region airbag and a massage unit even if the perineal region airbag for stimulating the perineal region is installed in a seat portion of a massager, which is designed to massage the back, the back and the thighs of a user while the massage unit moves from the backrest to the thigh portion of the massager.

### Technical Solution

To accomplish the above-mentioned objects, according to the present invention, there is provided a massager having a perineal region massage function including: a backrest frame supporting a user's back and having an opening formed to be open; a seat frame connected to the lower end of the backrest frame to support the buttocks and thighs of the user and having an opening communicating with the opening of the backrest frame; a massage unit for massaging the user's body part while moving along both sides of the openings of the backrest frame and the seat frame; an elastic band made of an elastic material, mounted to traverse the opening of the seat frame, and transferring pressure applied from the massage unit to the buttocks or thighs of the user through the opening of the seat frame; a perineal region airbag mounted on the elastic band to pressurize the user's perineal region upward while expanding by the air supplied from the outside; and an air supply unit for injecting air into the perineal region airbag and deflating the perineal region airbag.

The massager according to an embodiment of the present invention further includes a perineal region heater mounted on the upper surface of the perineal region airbag to generate heat by power applied from the outside.

The massager according to an embodiment of the present invention further includes a heater fixing clip for fixing the end of the perineal region heater to the end of the perineal region airbag.

The massager according to an embodiment of the present invention further includes: an anus region airbag mounted at the rear of the perineal region airbag to pressurize the user's anus region while expanding by air supplied from the outside; and an anus region heater mounted on the upper surface of the anus region airbag to generate heat by power applied from the outside.

The massager according to an embodiment of the present invention further includes a thigh airbag mounted on both sides of the front portion of the perineal region airbag, expanding by air supplied from the outside, and pressing the user's thighs upward.

Support pads are stacked on the lower surfaces of the perineal region airbag and the thigh airbag.

The support pads, the perineal region airbag, and the thigh airbag are embedded in an exterior cover made of a flexible material, and a plurality of pockets are provided in the exterior cover in a pouch shape of which one side is opened so that both ends of the thigh airbag are inserted and supported.

One end of the elastic band is fixed to one side of the seat frame by a screw, and the other end of the elastic band is fixed to the other side of the seat frame by a band coupling member, which includes a band connection ring around which the other end of the elastic band is wound and fixed and a connection bracket connected with the band connection ring and coupled to the other side of the seat frame by a screw.

The massager according to an embodiment of the present invention further includes: a vibrating protrusion protruding upward from an upper surface of the perineal region airbag; and

a vibration motor mounted inside the vibrating protrusion to generate vibration by a power applied from the outside.

A control method of the massager according to an embodiment of the present invention includes the steps of: (S1) massaging a user's body part while a massage unit moves along rails of a backrest frame and a seat frame; and (S2) when performing step (S1), expanding a perineal region airbag by supplying air to the perineal region airbag while the massage unit is located at the backrest frame, deflating and contracting the perineal region airbag when the massage unit lowers from the backrest frame, enters an opening of the seat frame and passes a predetermined point of the lower end of the backrest frame.

### Advantageous Effects

According to the present invention, the massager having a perineal region massage function includes the perineal region airbag, which is mounted on the seat portion to be expanded and contracted through supply and discharge of air, and the perineal region heater for providing heat, thereby improving the sexual function of a user and solving menopausal symptoms, chronic constipation, and the like by stimulating muscles of the perineal region and activating the blood circulation of the user.

Especially, since the perineal region airbag is stably supported on the highly elastic band crossing the opening of the seat frame, thereby applying pressure and heat to the perineal region, and performing an acupressure action of the buttocks and thighs by the massage unit without interruption.

In addition, in a case in which the anus region airbag is configured in parallel with the perineal region airbag, the massager according to the present invention can simultaneously and selectively press the perineal region and the anus region and provide heat to the perineal region and the anus region to stimulate them, thereby treating or preventing a disease of the anus region.

### DESCRIPTION OF DRAWINGS

FIG. 1 is a perspective view schematically illustrating a massager having a perineal region massage function according to an embodiment of the present invention.

FIG. 2 is an exploded perspective view illustrating a main configuration for performing a perineal region massage in the massager illustrated in FIG. 1.

FIG. 3 is a perspective view of a main configuration of the massager illustrated in FIG. 1.

FIGS. 4A and 4B are front views illustrating different embodiments of an airbag of the massager illustrated in FIG. 1.

FIG. 5 is an exploded perspective view illustrating an elastic band of the massager illustrated in FIG. 1.

FIG. 6 is a sectional view of the elastic band illustrated in FIG. 5.

FIG. 7 is a view illustrating a coupling structure of a perineal region airbag and a perineal region heater of the massager illustrated in FIG. 1.

FIG. 8 is a side-sectional view of the massager illustrated in FIG. 1.

FIG. 9 is a plan view illustrating a state in which main components of the massager illustrated in FIG. 1 are mounted on an external cover.

FIG. 10 is a sectional view of the main components of the massager illustrated in FIG. 9.

FIG. 11 is a plan view of a massager according to another embodiment of the present invention.

FIG. 12 is a sectional view taken along the line I-I of FIG. 11.

FIG. 13 is a perspective view of a massager according to another embodiment of the present invention.

### BEST MODE

There is a massager having a perineal region massage function including: a backrest frame supporting a user's back and having an opening formed to be open; a seat frame connected to the lower end of the backrest frame to support the buttocks and thighs of the user and having an opening communicating with the opening of the backrest frame; a massage unit for massaging the user's body part while moving along both sides of the openings of the backrest frame and the seat frame; an elastic band made of an elastic material, mounted to traverse the opening of the seat frame, and transferring pressure applied from the massage unit to the buttocks or thighs of the user through the opening of the seat frame; a perineal region airbag mounted on the elastic band to pressurize the user's perineal region upward while expanding by the air supplied from the outside; and an air supply unit for injecting air into the perineal region airbag and deflating the perineal region airbag.

### MODE FOR INVENTION

The objects, features, and advantages of the present invention described above will become more apparent through the following embodiments in connection with the accompanying drawings.

The following specific structure to functional descriptions are illustrated for the purpose of describing an embodiment according to the concept of the present invention, and embodiments according to the concept of the present invention may be implemented in various forms and should not be construed as being limited to the embodiments described herein or the application.

The embodiments according to the concepts of the present invention may apply various changes and may have various forms, and thus certain embodiments are illustrated in the drawings and will be described in detail in the specification or application. However, it should be understood that the embodiments of the present invention are not intended to limit the embodiments of the present invention to a specific disclosure, and include all modifications, equivalents, and replacements included in the spirit and technical scope of the present invention.

Terms such as first and/or second may be used to describe various components, but the components are not limited to the terms. The terms are only for the purpose of distinguishing one component from other components, for example, without departing from the scope of the present invention, a first component may be referred to as a second component, and similarly, a second component may also be referred to as a first component.

It should be understood that when a component is referred to as being “connected” or “coupled” to another component, it may be directly connected or coupled to the other component, but it should be understood that there may be other components therebetween. On the other hand, when a component is referred to as being “directly connected” or “directly coupled” to another component, it should be understood that there is no other component therebetween. Other expressions for describing the relations between the components, that is, the expression “between” and “directly between” or “adjacent to” and “directly adjacent to” should be interpreted in the same manner.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

Unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

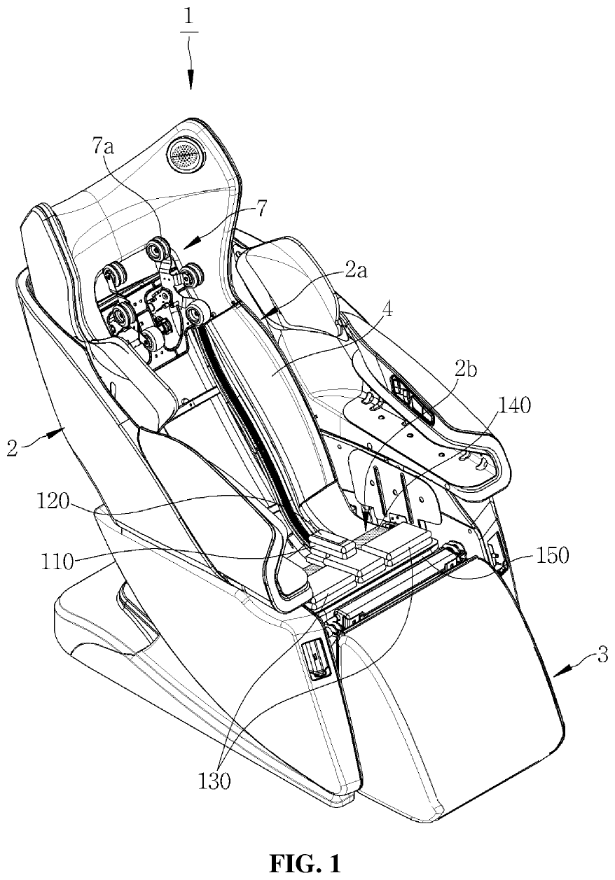

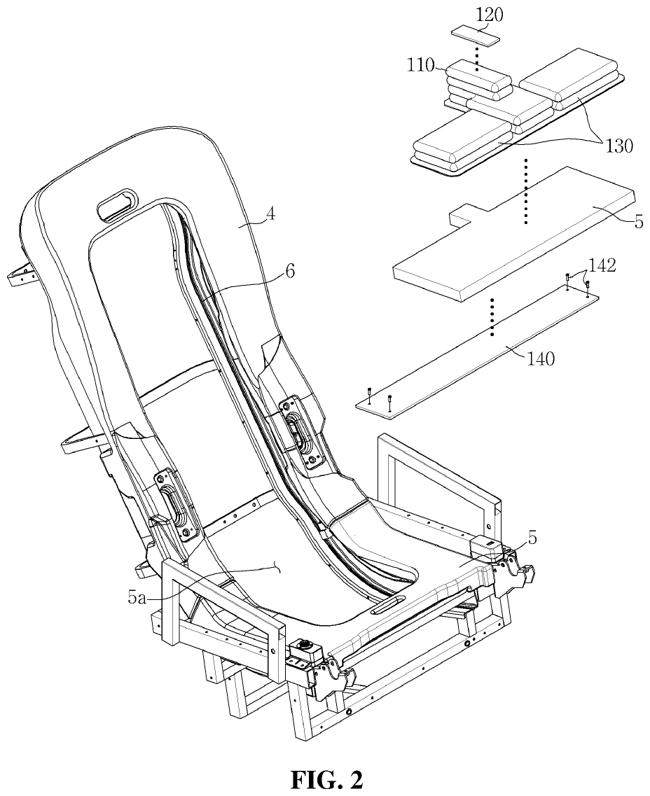

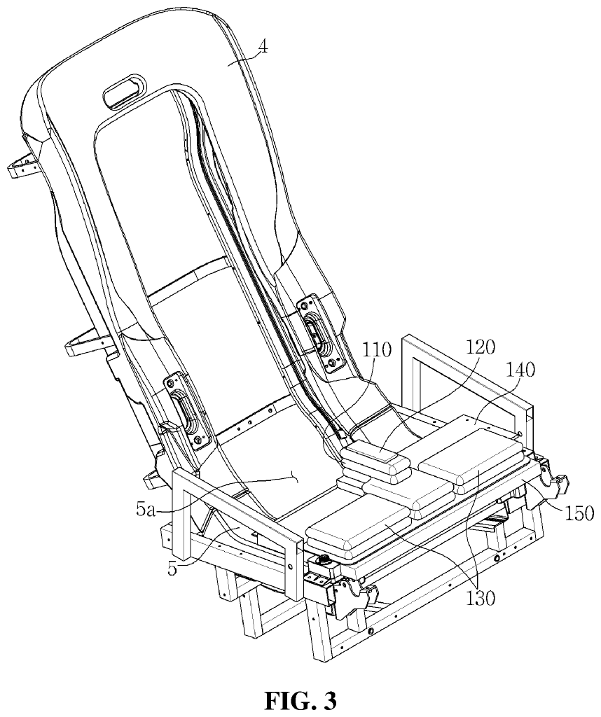

Referring to FIGS. 1 to 3, a massager 1 may include a body massage unit 2 forming an area for accommodating at least a portion of a body of a user and massaging the body of the user, and a leg massage unit 3 massaging the user's legs.

The body massage unit 2 includes a backrest portion 2a supporting the back portion of the user while the user sits thereon, a seat portion 2b supporting the buttocks and the thighs of the user. The backrest portion 2a and the seat portion 2b are respectively provided with a backrest frame 4 and a seat frame 5 for mounting and supporting various components such as a massage unit 7 for supporting the user's load and performing massage. An opening is formed in the middle of the backrest frame 4 and the seat frame 5, and rails 6 are mounted at both sides of the opening to be extended vertically. The backrest frame 4 and the seat frame 5 include an interior material made of urethane foam, sponge or expanded polystyrene and an exterior cover made of a polyester-based raising tricot, synthetic leather, natural leather, or the like to comfortably support the user's body parts.

The massage unit 7 providing a mechanical stimulation to the user is mounted at the rear portion of the backrest frame 4 and the seat frame 5 to massage the back, the waist, the buttocks, and the thighs of the user while vertically moving along the rails 6. The massage unit 7 has a sample member 7a capable of providing mechanical stimulations, such as ‘tapping’ or ‘rubbing’ by applying vibration and pressure to various parts of the user's body. The sample member 7 a may be a roller or a ball.

The seat frame 5 is connected to the lower end of the backrest frame 4 to support the buttocks and thighs of the user. In order for the massage unit 7 to press and massage the buttocks and thighs of the user, an opening 5a is formed to be vertically opened in the middle of the seat frame 5.

A perineal airbag 110 and a thigh airbag 130 for pressing and massaging upward the perineal region and the thigh region of the user sitting on the massager are respectively mounted at the approximately central portion and the front portion of the seat frame 5. The thigh airbag 130 is disposed to traverse the front end of the seat frame 5 left and right, and the perineal airbag 110 is disposed behind the central portion of the thigh airbag 130. The perineal airbag 110 and the thigh airbag 130 may be integrally formed, but their spaces may be formed to be separated from each other so that the perineal airbag 110 and the thigh airbag 130 can be independently expanded and contracted.

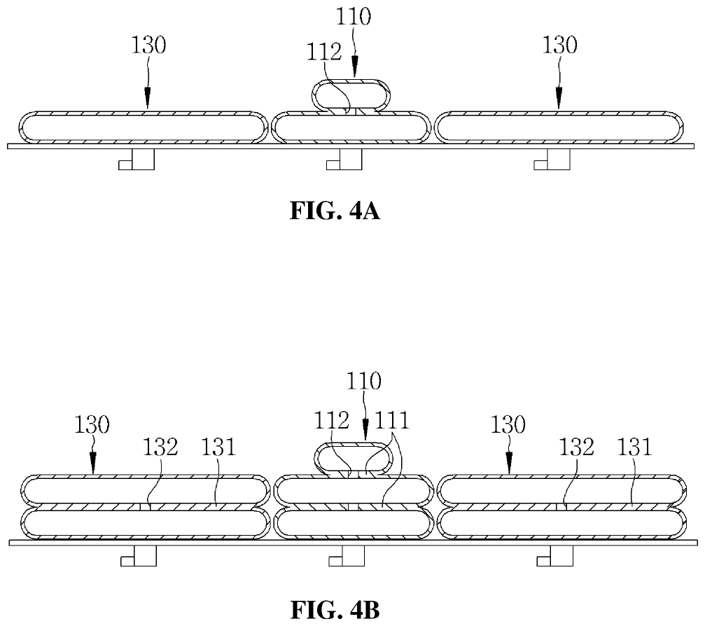

As illustrated in FIG. 4A, the perineal airbag 110 and the thigh airbag 130 may be configured in one stage in the vertical direction, but may be formed only in multiple stages as illustrated in FIG. 4B. In a case in which the perineal airbag 110 and the thigh airbag 130 are formed in multiple stages, the perineal airbag 110 and the thigh airbag 130 are divided in multiple stages by partition walls 111 and 131 having holes 112 and 132 through which air passes. Moreover, since the outer surfaces of the perineal airbag 110 and the thigh airbag 130 are formed in a bellows shape, they are expanded in a vertical direction when air is injected, thereby maximizing acupressure stimulation to the perineal region and the thigh of the user.

The perineal airbag 110 and the thigh airbag 130 may be expanded or contracted while air is injected or discharged by an air supply unit (not shown) formed at a predetermined portion of the massager 1, thereby pressurizing or releasing upward the perineal region and thighs of the user. The air supply unit may be configured by applying a known air injection and discharge device such as an air compressor.

The perineal airbag 110 is mounted and supported on the elastic band 140 mounted on the opening 5a of the seat frame 5. The elastic band 140 is made of an elastic material such as rubber or silicone, is formed in a laterally long flat plate shape such that both ends of the elastic band 140 are fixed to both ends of the seat frame 5 by fixing means such as screws 142. The elastic band 140 is mounted to horizontally traverse the opening 5a of the seat frame 5 to support the perineal airbag 110 and to transfer pressure applied from the sample member 7a of the massage unit 7 to the buttocks or thighs of the user through the opening 5a of the seat frame 5. That is, the elastic band 140 functions to support the perineal airbag 110 and to smoothly transfer pressure of the massage unit 7 to the body part (buttocks and thighs) of the user.

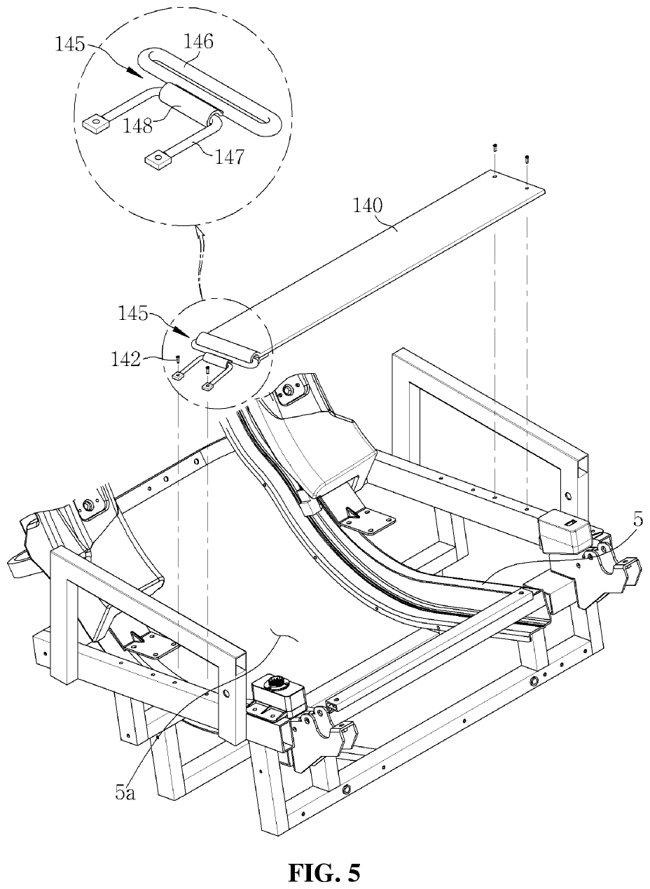

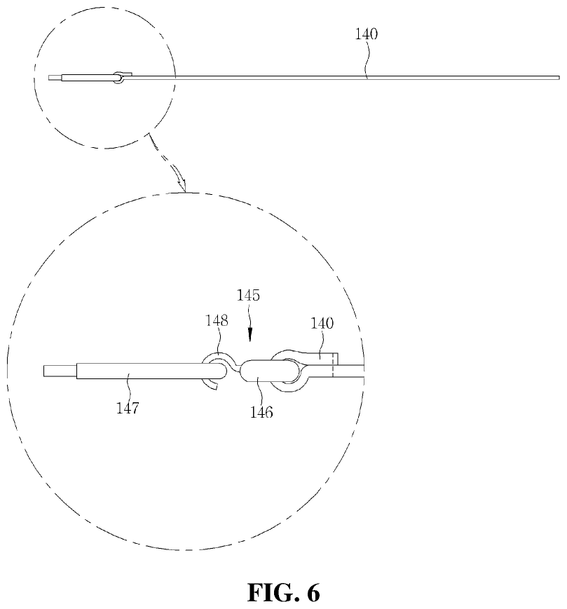

Both ends of the elastic band 140 may be fixed to both ends of the seat frame 5 by screws 142. However, as illustrated in FIGS. 5 and 6, one end of the elastic band 140 may be fixed to one side of the seat frame 5 by the screw 142, and the other end of the elastic band 140 may be fixed to the other side of the seat frame 5 by a band coupling member 145. The band coupling member 145 includes a band connection ring 146, which is in the shape of a long hole elongated in the back-and-forth direction, and around which an end of the elastic band 140 is wound and fixed, and a “ [symbol] ”-shaped connection bracket 147 which is connected to the band connection ring 146 and is screw-coupled to the other side of the seat frame 5. A hook portion 148 that is caught and connected to the end portion of the connection bracket 147 is formed integrally with the band connection ring 146.

Accordingly, the connection bracket 147 is fixed at an end of the seat frame 5 by a screw 142, an end of the elastic band 140 is inserted and sewed into the band connection ring 146, and then, the elastic band 140 is pulled toward the connection bracket 147 so that a predetermined tensile force is applied to the elastic band 140. In the state in which the predetermined tensile force is applied to the elastic band 140, the hook portion 148 of the band connection ring 146 is caught by the connection bracket 147 so that the elastic band 140 is mounted. In this instance, as described above, since the elastic band 140 may be fixed by applying a predetermined tensile force to the elastic band 140, when the perineal airbag 110 expands to apply pressure upward, the perineal airbag 110 may more elastically support the perineal airbag 110, thereby preventing the loss of the pressing force.

Referring back to FIGS. 1 to 3, the thigh airbag 130 is supported on the front end of the seat frame 5 outside the front portion of the elastic band 140.

In addition, a support pad 150 such as a sponge may be stacked on the lower surfaces of the perineal airbag 110 and the thigh airbag 130.

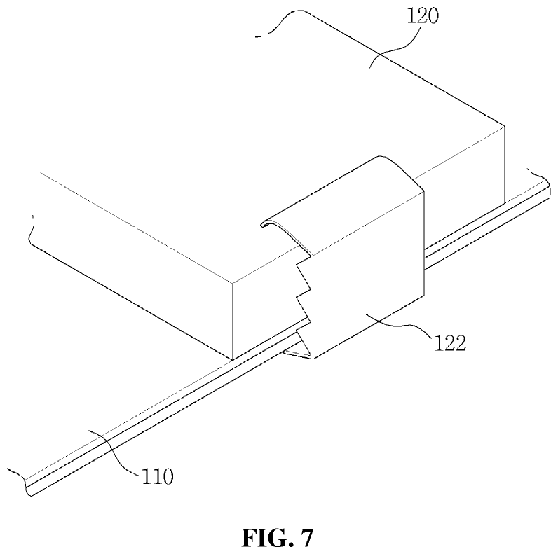

A perineal region heater 120 for providing heat to the perineal region is mounted on the upper surface of the perineal airbag 110. The perineal region heater 120 has a structure in which a heating wire is mounted in a planar heater of a rectangular pad shape or in a rectangular thermal conductive pad, and generates heat by power applied from a controller of the massager 1 to provide heat to the perineal region of the user. The perineal region heater 120 may be adhered to the upper surface of the perineal airbag 110 by an adhesive. In a case in which the perineal airbag 110 repeats expansion and contraction, adhesive strength of the adhesive may be weakened and the perineal region heater 120 may be separated from the upper surface of the perineal region airbag 110. In order to prevent separation of the perineal region heater 120, as illustrated in FIG. 7, the end of the perineal region heater 120 may be firmly fixed to the end of the perineal airbag 110 by using a heater fixing clip 122. The heater fixing clip 122 is a substantially “ [symbol] ”-shaped clip of which the upper end and the lower end are curved. After the end of the perineal region heater 120 and the end of the perineal airbag 110 are inserted between the upper end and the lower end of the heater fixing clip 122, when the upper end and the lower end of the heater fixing clip 122 are closed up by a tool, the heater fixing clip 122 firmly fixes the end of the perineal region heater 120 and the end of the perineal airbag 110.

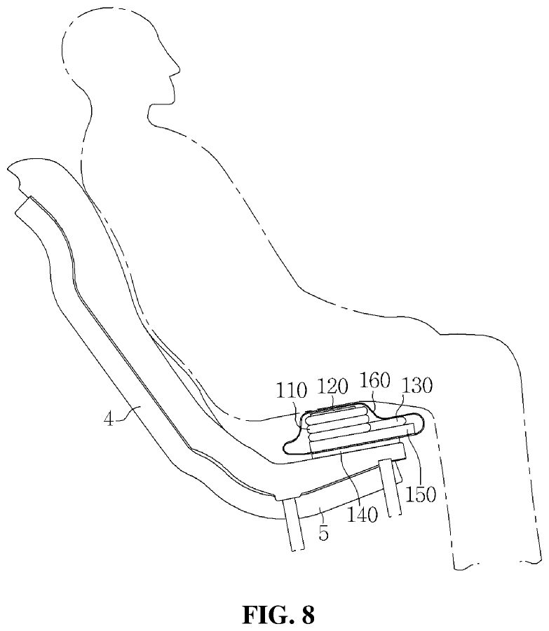

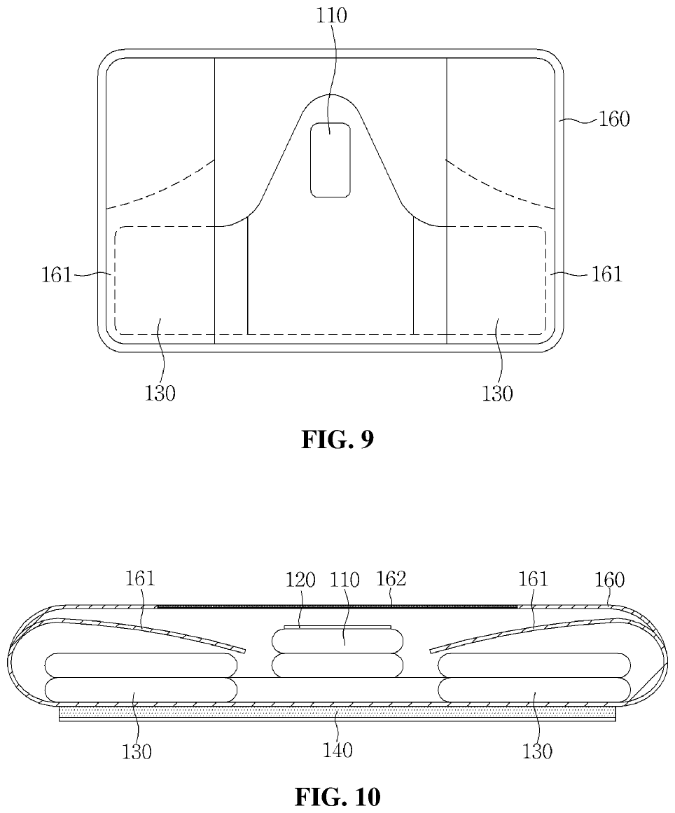

As illustrated in FIGS. 8 to 10, the perineal airbag 110, the perineal region heater 120, the thigh airbag 130, and the support pad 150 are embedded in a pocket-shaped exterior cover 160 made of a flexible material such as leather or cloth. In order to allow the thigh airbag 130 to maintain a normal position without moving inside the external cover 160, the outer cover 160 may be provided with a plurality of pockets 161 formed in a pouch shape of which one side is open such that both ends of the thigh airbag 130 are inserted and supported.

The external cover 160 is disposed outside the massager 1 to comfortably support the buttocks and thighs of the user, and is made of a flexible material capable of smoothly transferring pressure caused by the expansion of the perineal region airbag 110 and the thigh airbag 130 to the perineal region and thighs of the user.

The upper surface of the external cover 160 may be formed of a mesh sheet 162 to more smoothly transfer the pressure caused by the expansion of the perineal airbag 110 and the thigh airbag 130.

As described above, in a case in which the user selects a perineal region massage mode on an input unit (not shown) to operate the massager 1 after sitting on the seat portion 2b (see FIG. 1), air is supplied from an air supply unit (not shown) to the perineal region airbag 110 such that the perineal region airbag 110 expands to pressurize the perineal region of the user upward, and the controller of the massager 1 applies power to the perineal region heater 120 to generate heat from the perineal region heater 120, thereby providing heat to the perineal region. When the perineal region massage function is performed by the operation of the perineal airbag 110 and the perineal region heater 120, the massage unit 7 of the massager 1 may simultaneously perform a function of massaging the back, the waist, the buttocks, and the thighs of the user while moving along the rails 6 mounted on the backrest frame 4 and the seat frame 5.

In this instance, in a case in which the massage unit 7 performs acupressure and massage on the buttocks and thighs in a state in which the perineal airbag 110 is expanded, the massage unit 7 interferes with a portion (an edge portion) of the perineal region airbag 110, and it may cause a damage of the perineal region airbag 110 or deterioration of durability. In order to prevent such a problem, the air supply unit supplies air to the perineal region airbag 110 to expand the perineal region airbag 110 while the massage unit 7 of the massager 1 is located outside the opening 5a of the seat frame 5, namely, while the massage unit 7 is located on the backrest frame 4, deflates the perineal region airbag 110 so that the perineal region airbag 110 is contracted when the massage unit 7 lowers from the backrest frame 4 and enters the opening 5a of the seat frame 5, namely, when the massage unit 7 passes a point (e.g., a curved point where the backrest frame 4 and the seat frame 5 are connected to each other) predetermined at the lower end of the backrest frame 4. That is, the air supply unit controls supply and discharge of air.

In other words, the massage unit 7 controls the operation of the air supply unit to discharge air from the perineal region airbag 110 so as to contract the perineal region airbag 110 directly before the massage unit 7 lowering along the backrest frame 4 enters the opening 5a of the seat frame 5 during the process that the massage unit 7 provides a massage function to the body parts of the user while moving along the rails 6 mounted on the backrest frame 4 and the seat frame 5.

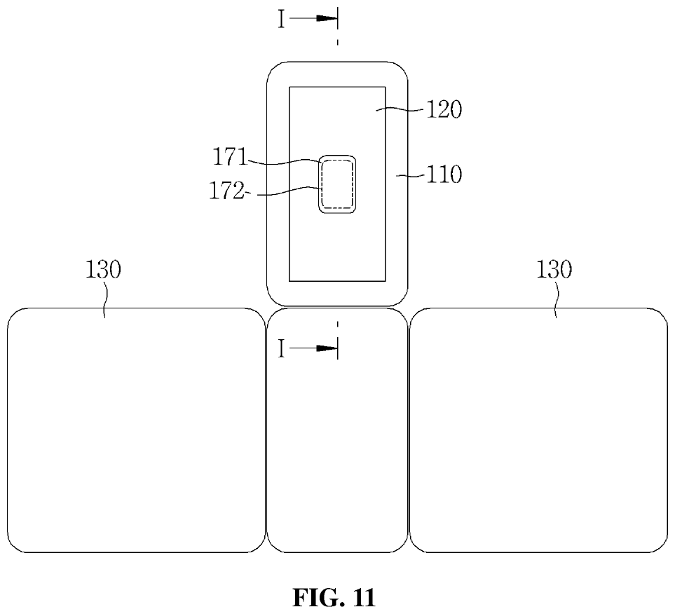

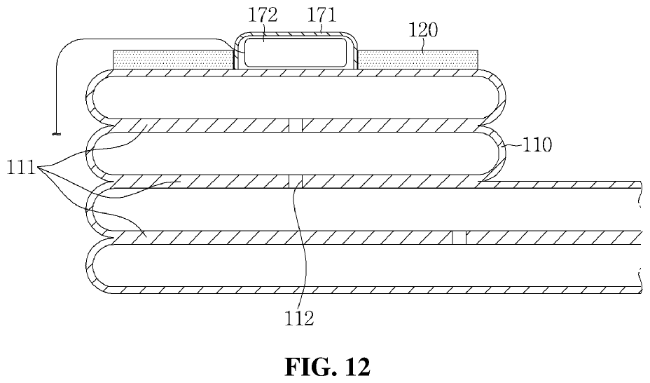

FIGS. 11 and 12 illustrate another embodiment of the present invention, in which the massager includes a vibrating protrusion 171 protruding upward from an upper surface of the perineal region airbag 110, and a vibration motor 172 mounted inside the vibrating protrusion 171 to generate vibration by a power applied from the controller of the massager 1. When the vibrating protrusion 171 and the vibration motor 172 are mounted together with the perineal region heater 120 on the upper portion of the perineal region airbag 110, it is possible to provide pressure and heat to the perineal region of the user and apply vibration to further stimulate the muscle of the perineal region.

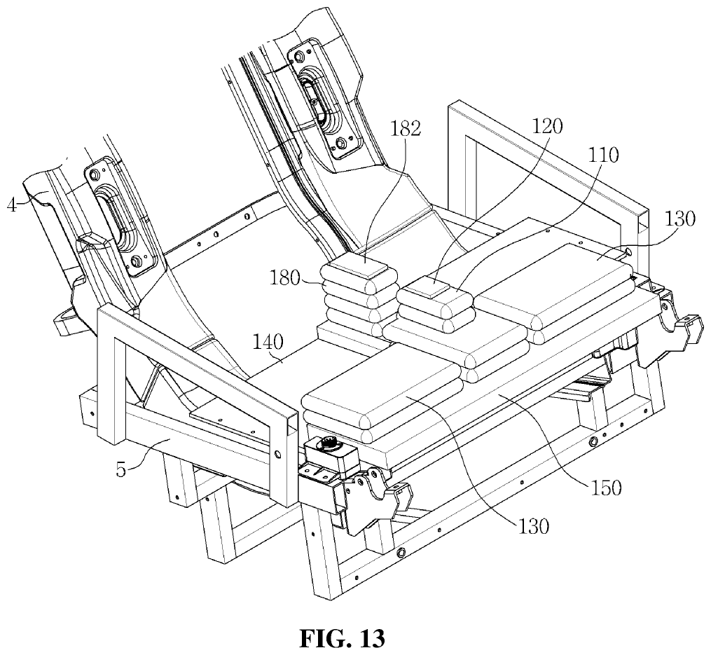

In addition, FIG. 13 illustrates another embodiment of the present invention, wherein the massager of this embodiment further includes an anus airbag 180 for pressing the anus region of the user at the rear of the perineal airbag 110. The anus airbag 180 is expanded by the air supplied from the air supply unit (not shown) to pressurize and contract the anus region of the user.

A rectangular pad-shaped anus heater 182 of a rectangular pad shape that generates heat by power applied from the outside may be mounted on the upper surface of the anus airbag 180.

According to the present invention, the massager having a perineal region massage function includes the perineal region airbag, which is mounted on the seat portion to be expanded and contracted through supply and discharge of air, and the perineal region heater for providing heat, thereby improving the sexual function of a user and solving menopausal symptoms, chronic constipation, and the like by stimulating muscles of the perineal region and activating the blood circulation of the user.

Especially, since the perineal region airbag is stably supported on the highly elastic band crossing the opening of the seat frame, thereby applying pressure and heat to the perineal region, and performing an acupressure action of the buttocks and thighs by the massage unit without interruption.

In addition, in a case in which the anus region airbag is configured in parallel with the perineal region airbag, the massager according to the present invention can simultaneously and selectively press the perineal region and the anus region and provide heat to the perineal region and the anus region to stimulate them, thereby treating or preventing a disease of the anus region.

As illustrated in the drawings, since the perineal region airbag 110 and/or the anus region airbag 180 may be formed in a bellows shape, they are expanded in a vertical direction when air is injected, thereby maximizing acupressure stimulation to the perineal region and the anus region of the user.

Although preferred embodiments of the present invention have been described above, the present invention may use various changes, modifications, and equivalents. It is clear that the present invention can be suitably applied by appropriately modifying the embodiments. Therefore, the description of the present invention does not limit the scope of the present invention determined by the limitation of the following claims.

1. A massager having a perineal region massage function, the massager comprising:
a backrest frame supporting a user's back and having an opening formed to be open;
a seat frame connected to the lower end of the backrest frame to support the buttocks and thighs of the user and having an opening communicating with the opening of the backrest frame;
a massage unit for massaging the user's body part while moving along both sides of the openings of the backrest frame and the seat frame;
an elastic band made of an elastic material, mounted to traverse the opening of the seat frame, and transferring pressure applied from the massage unit to the buttocks or thighs of the user through the opening of the seat frame;
a perineal region airbag mounted on the elastic band to pressurize the user's perineal region upward while expanding by the air supplied from the outside; and
an air supply unit for injecting air into the perineal region airbag and deflating the perineal region airbag.

2. The massager according to claim 1, further comprising:
a perineal region heater mounted on the upper surface of the perineal region airbag to generate heat by power applied from the outside.

3. The massager according to claim 2, further comprising:
a heater fixing clip for fixing the end of the perineal region heater to the end of the perineal region airbag.

4. The massager according to claim 1, further comprising:
an anus region airbag mounted at the rear of the perineal region airbag to pressurize the user's anus region while expanding by air supplied from the outside; and
an anus region heater mounted on the upper surface of the anus region airbag to generate heat by power applied from the outside.

5. The massager according to claim 1, further comprising:
a thigh airbag mounted on both sides of the front portion of the perineal region airbag, expanding by air supplied from the outside, and pressing the user's thighs upward.

6. The massager according to claim 1, wherein support pads are stacked on the lower surfaces of the perineal region airbag and the thigh airbag.

7. The massager according to claim 6, wherein the support pads, the perineal region airbag, and the thigh airbag are embedded in an exterior cover made of a flexible material, and a plurality of pockets are provided in the exterior cover in a pouch shape of which one side is opened so that both ends of the thigh airbag are inserted and supported.

8. The massager according to claim 1, wherein one end of the elastic band is fixed to one side of the seat frame by a screw, and the other end of the elastic band is fixed to the other side of the seat frame by a band coupling member, which includes a band connection ring around which the other end of the elastic band is wound and fixed and a connection bracket connected with the band connection ring and coupled to the other side of the seat frame by a screw.

9. The massager according to claim 1, further comprising:
a vibrating protrusion protruding upward from an upper surface of the perineal region airbag; and
a vibration motor mounted inside the vibrating protrusion to generate vibration by a power applied from the outside.

10. A control method of the massager according to claim 1, the control method comprising:
massaging a user's body part while a massage unit moves along rails of a backrest frame and a seat frame; and
when performing the massaging, expanding a perineal region airbag by supplying air to the perineal region airbag while the massage unit is located at the backrest frame, deflating and contracting the perineal region airbag when the massage unit lowers from the backrest frame, enters an opening of the seat frame and passes a predetermined point of the lower end of the backrest frame.
